(19) United States
(12) Patent Application Publication
(10) Pub. No.: US 2008/0009752 A1
(43) Pub. Date: Jan. 10, 2008

(54) System for Cardiovascular Data Display and Diagnosis

(76) Inventor: Michael Henry Butler, St. Simons Island, GA (US)

Correspondence Address: OPPENHEIMER WOLFF & DONNELLY LLP, 45 SOUTH SEVENTH STREET, SUITE 3300, MINNEAPOLIS, MN, 55402, US

(21) Appl. No.: 11/774,479

(22) Filed: Jul. 6, 2007

Related U.S. Application Data
(60) Provisional application No. 60/819,033, filed on Jul. 7, 2006.

(51) Int. Cl. A61B 5/02

(52) U.S. Cl. 600/481

(57) ABSTRACT

To provide a tool for the efficient and collaborative treatment of cardiovascular disease, a comprehensive report is created which includes clinical data, ECG data and ECHO data, all presented in a comprehensive format for the easy review an analysis by care givers. The systems are configured to deal with the different data formats involved and subsequently generate the reports in a manner that specifically allows for ease of use. Further, the format of the report is specifically conducive to the transmission of this data over networks to allow remote analysis and evaluation by specialists, including cardiologists.

### CROSS REFERENCE TO RELATED APPLICATIONS

This application is related to Provisional Application Ser. No. 60/819,033, filed on Jul. 7, 2006, entitled SYSTEM FOR CARDIOVASCULAR DATA DISPLAY AND DIAGNOSIS, which is relied upon for priority and which is incorporated herein in its entirety.

### BACKGROUND

The present invention relates to the treatment of cardiovascular disease. More specifically, the present invention provides a system for the collection and organized management of cardiovascular related data so it may be effectively and efficiently used by health care professionals.

Cardiovascular disease is a common health problem and poses an increasing demand on the health care system as the population ages. Cardiovascular diseases are often treated by both primary care physicians (“PCPs”, i.e., internists and family practitioners) and cardiologists. Screening methods used to detect cardiovascular disease include stress testing (either treadmill or chemical) paired with other cardiac imaging technology, such as electrocardiogram (“ECG”), echocardiogram (“ECHO”), or nuclear imaging.

Primary care physicians and cardiologists screen for cardiovascular disease differently due largely to different levels of experience, resources and training. Primary care physicians typically use stress ECGs alone while, cardiologists typically use stress ECGs combined with ECHOs. The additional use of ECHO offers a cardiologist superior diagnostic specificity, thus providing the opportunity for superior patient care. From a pure business perspective, the additional use of ECHOs also provides an additional service to the patient, thus generating further revenue to the cardiologist

As mentioned above, primary care physicians typically utilize ECGs when dealing with cardiovascular disease. These primary care physicians are trained and familiar with ECGs, thus providing them with a familiar tool. In addition, ECG equipment is typically available to the primary care physician. Primary care physicians however are typically not trained to interpret ECHO studies. Naturally, some primary care physicians may have some experience in dealing with ECHO studies, however they are not typically trained at the same level as cardiologists. For this reason, patients are typically referred to cardiologists for detailed analysis of ECHO studies and results.

In the treatment of patients with cardiovascular disease, primary care physicians and cardiologists are often required to combine their experiences and skills to provide the best care. However, the management and organization of data to most effectively utilize the combined skills of these individuals is challenging. In addition, the cardiologist and the primary care physicians are often located in different facilities, thus requiring the transfer of files and/or data between locations. Naturally, this creates complications and challenges in the handling of information.

To effectively utilize multiple care resources, telemedicine is used today, but mainly in the field of radiology. U.S. Pat. No. 5,469,353 outlines a method of having radiologic images digitized and then routed through a telecommunications network to a remote location for interpretation and reporting. The '353 patent describes the transfer of conventional radiologic studies (X-ray, CT, MRI) from one radiology center to another radiology center.

Telemedicine has been more difficult to implement in cardiology because of limiting technology. Echocardiography and cardiac catheterization images are moving pictures when interpreted. As is well known, there is a much greater amount of digital information in a moving image than a static one thus making it challenging to transfer data electronically. Prior to the advent of broadband technology timely transfer of these data sets to a remote location was not practical. Even using broadband, the transfer of large files can be a challenge.

As another example of telemedicine, U.S. Pat. No. 5,715,823 describes a method of transferring ultrasound diagnostic images over a data communications network for remote interpretation and reporting. This patent describes real time transfer of images between remote locations. The system highlighted in this patent allows a more experienced echo cardiographer to manipulate the controls (contrast, gain, etc. . . . ) on a remote machine while someone else manipulates the probe. The '823 patent enhances an existing service by allowing a limited level of remote operation, but does not bring a new service to the remote location. The '823 patent further outlines a method of combining the ultrasound images and reports for remote interpretation. This combination is fairly straightforward as it involves the combination of common types of images (e.g. the combination of the two ultrasound images).

Given the challenges listed above, there is a need to provide a mechanism for the efficient sharing of data so cardiovascular disease can be treated. This includes a need to transfer information/data between health care professionals for the effective treatment of cardiovascular conditions.

### SUMMARY OF THE INVENTION

The system and methods of the present invention will make available superior cardiac technology to a larger group of cardiovascular patients, and will combine previously separate reports to provide a new diagnostic tool. In addition, the system and method of the present invention will provide tools and resources to the primary care physician that will allow them to offer more comprehensive care to their patients.

Generally speaking, the present invention provides at least the following features and advantages: (1) Reformats existing technology so that cardiovascular imaging data can be easily transmitted, interpreted and reported over the internet; (2) Provides changes to the cardiovascular imaging market by providing additional tools and resources; (3) Offers a staffing service contract to PCPs thus giving them access to ECHO technicians trained to supervise and interpret stress ECHOs; (4) Creates a potential new position that combines the skills of an echo cardiographer and an exercise technologist (5) Offers a subscription service to cardiologists that would give them access to studies done in PCPs offices. The system outlined below will provide a new service to cardiovascular patients and primary care physicians (PCP) thus allowing for better patient care. When implemented, a PCP will be able to provide the more accurate stress ECHO in his office while all portions of the test; stress, ECG, and ECHO will be interpreted remotely by a cardiologist.

A stress ECHO typically consists of three different data sets: clinical data, ECG data, and ECHO data. The clinical data includes the patient's age, medical history, blood pressure, and their performance on the treadmill. This is recorded by the technologist performing the test. The stress ECG data is recorded by the stress machine and is graphically presented on paper as five to ten serial ECGs. Lastly, the ECHO data is made up of video images of the heart, recorded by the ECHO machine and interpreted on VHS tape or digitally on a computer. The present invention provides for the integration of these data sets for easy interpretation and reporting, either at the originating site or a remote location.

As known by those in the industry, cardiovascular ECHO images are obtained digitally by ultrasound equipment. This data can then be reformatted into DICOM by a picture archiving and communication system (PACS). DICOM is an industry standard format that allows the information to be more easily stored or transferred over the internet. The fact that ECHO pictures are moving (i.e. video or many pictures) rather than static (one picture) means that the ECHO data set is large. Broadband internet access and a PACS will be needed to send and receive this data.

Unfortunately stress ECG data is not standardized across the industry. Most are now digital and the data sets are relatively small. Due to their size alone, ECG data sets may be somewhat easily transmitted between various locations.

While specific practices may vary amongst individuals, a cardiologist will typically analyze stress ECHOs by first reading the clinical data on the front sheet of the patient's stress chart. Secondly, a cardiologist interprets a series of ECGs. And finally, the cardiologist reviews the ECHO images on VHS tape or a PACS reading station and interprets them. Using information from the cardiologist, a final report is then generated by the ECHO tech either typing or dictating the results from each data set into one integrated report. (FIG. 1 illustrates one example of a final report that is typically generated.)

The present invention provides all of this data digitally and presents the data simultaneously on one computer reading station that can be local or remote. This enables a cardiologist to select a patient on the computer display, review the patient's clinical, ECG, and ECHO data simultaneously, and finalize the tech's report. Collecting the data in this format allows for great flexibility in the care of patients as the information is more easily shared.

Most PCPs already perform stress tests in their office but do not provide ECHO services. The present invention will allow PCP the ability to provide patients with this additional diagnostic tool. Adding ECHO and stress ECHO represents no increase in liability as a stress ECHO is no greater risk than a stress ECG. As suggested above, to enable the present invention, the PCPs will need updated stress equipment, an ECHO machine with a limited PACS, and an ECHO tech or service contract. The expense of adding this equipment is easily justified however by allowing the PCPs to provide a higher level of cardiac care for patients and an additional revenue stream for the practioner. A practice of four PCP's could easily generate the volume of enhanced ECHO based services to justify the additional expense.

Stress lab staffing is a critical issue. Two ACLS certified people must be in the room to efficiently operate. An efficient staffing model includes a registered nurse (RN) and an ECHO tech, both trained in stress testing. Typically, primary care physicians that do stress testing already employ a qualified RN. Thus, a PCP may only need to hire an ECHO tech to implement and take advantage of the present invention.

Poorly trained personnel increase the risk of the procedure and the liability of the PCP. If an ECHO tech resigns, lab downtime will significantly impact the PCPs return on investment (ROI) in the above referenced additional equipment. As one other alternative to address this staffing issue, the sale of an ECHO machine to a PCP group under the present invention may come with an optional service contract. The service contract would provide an ECHO tech and, if needed, an RN with extensive training in stress testing. A staffing service contract will provide insurance against idle equipment and increased liability. The commonality of data as formatted and the ability to easily transit data to remote locations allows this staffing contract to be easily fulfilled. Down time is avoided by providing outsourcing opportunities, thus allowing more physicians access to comprehensive cardiovascular tools.

As mentioned above, primary care physicians currently do not employ ECHO techs. They also supervise their own stress tests which is generally an inefficient practice. To most efficiently utilize their time the physician should see patients in the clinic but is not required to supervise a stress test. Moving between the clinic and the stress lab slows patient flow in both areas. Changing this practice under the present invention will greatly increase the volume of the stress lab, and its efficiency. From a business perspective, this will also increase the return on investment to the stress lab.

The present invention also has the potential to create a new position or occupation (cardiovascular technologist) that combines the skill sets of echo cardiographers and exercise technologists. The ECHO tech needs to supervise and interpret the stress ECHO but they are not trained in stress testing. Under the present invention, the ECHO techs are further trained in stress testing to ensure the supply of reliable information to the PCPs. The cost of this training can be offset by the PCP service contract and ECHO tech student tuition. Motivated ECHO techs will be willing to do extra training enabling them to demand a higher salary.

The present invention further enables increased revenue for PCPs and efficiently utilizes the skills of cardiologists. As may be expected, primary care physicians performing cardiovascular imaging studies will negatively impact cardiologist's volume of work. A subscription service under the present invention offers cardiologists the ability to easily access and interpret PCP's studies. While several arrangements are possible, the cardiologist will likely charge a professional fee for this service. The additional professional fees would help offset the cardiologist's lost technical fees. In addition, this provides the primary care physician with the ability to incorporate cardiologist insight into the care provided to patients. Overall, this results in a much improved care system.

### BRIEF DESCRIPTION OF THE DRAWINGS

Further objects and advantages of the present invention can be seen by referring to the following detailed description, in conjunction with the drawings in which:

FIG. 1 illustrates an exemplary cardiac report of the prior art;

FIG. 2 illustrates a flow chart for the process utilized for the present invention to comprehensively collect cardiovascular data;

FIG. 3 is an example comprehensive report generated by the process and system of the present invention; and

FIG. 4 is a system diagram illustrating the components making up a typical cardiovascular data management system of the present invention.

### DETAILED DESCRIPTION OF THE INVENTION

As summarized above, the present invention provides a comprehensive system and method for collecting and reporting cardiovascular data in an organized and efficient manner. To provide more complete tools in the treatment of cardiovascular disease, the combined reports of the present invention include ECG data, ECHO data, and clinical data collected from various sources. Most significantly, all this data is combined into a single report and presented in a manner which is easily usable by all necessary parties. It is contemplated that the various parties involved include the primary care physician, a cardiologist, and multiple technicians and support staff individuals.

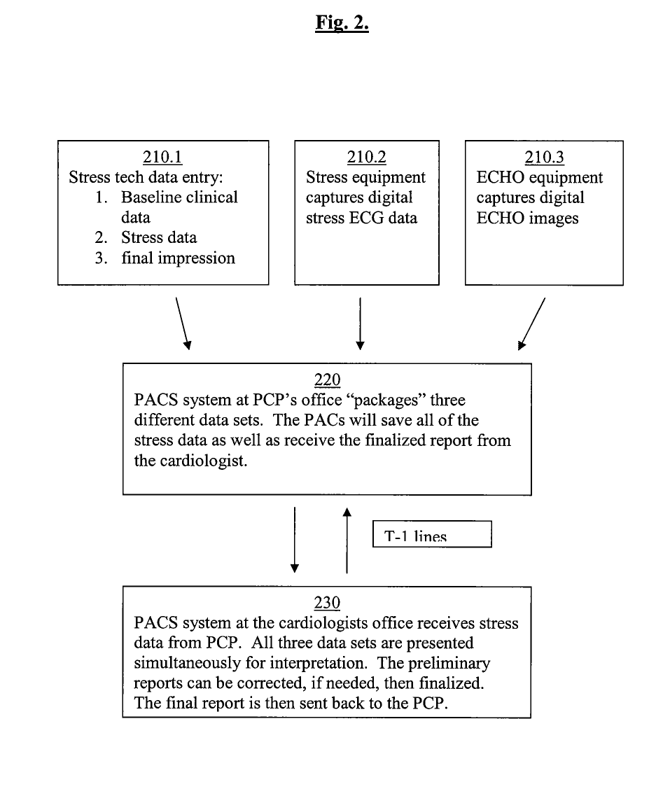

One embodiment of the process of the present invention is generally illustrated in FIG. 2. As shown, the process begins with data collection step 210, in which various portions of this step will occur separately. Generally, in step 210 a patients' clinical data, ECG data, and ECHO data will be collected separately from the appropriate sources. These separate data sets will then be integrated and reformatted for internet transfer in step 220. In this embodiment an integrated patient report is generated which includes each data set. Using this integrated patient report, a cardiologist may then review and report the patient data using a PACS (see, step 230). Such review can be done locally or remotely.

Currently there is no formal training program for a general cardiovascular technician. A training program for cardiovascular studies similar to radiology tech training where all different modalities are learned. A radiology tech is trained in X-ray, MRI, CT, ultrasound, and angiography. In cardiology there is separate training for ECHO, stress, nuclear, and catheterization techs. All cross training occurs Ad Hoc on the job. In this specific invention an ECHO tech would require additional training in stress testing. One natural result of the system outlined above is the development of a formal general cardiovascular technician training program.

As mentioned above, the data collection step 210 involves various portions. Each of these sub portions or subtasks may be carried out and parallel, where appropriate. The tech data entry step 210.1 involves data entry, typically carried out by a technician. Generally speaking this involves the collection of clinical data, such as baseline clinical data, stress data, and overall impressions. This data is typically maintained in a text format, which may be collected by data entry or through coordination with additional stored systems. For example, general background and data related to the patient may be obtained through clinic patient records etc.

In addition to the clinical data, the present invention also utilizes ECG data which is typically captured during stress testing. As shown in FIG. 2, at step 210.2 this ECG data is captured. As will be appreciated, this data is collected from ECG equipment, which produces an output which can be captured by further systems. This ECG data is typically graphical in nature, and may be produced at a paper output, however an electronic version of these graphical results is preferable. For example, equipment may be configured to include a graphical “capture” of a display image showing ECG data.

In addition to the data collection steps outlined above, step 210.3 captures ECHO images during patient stress testing. These ECHO images can be captured during the same stress testing steps preformed when ECG data is collected. As such, the collection of ECHO and ECG data is typically carried out at the same time, however using separate pieces of equipment. As can be appreciated, the ECHO data includes video images or moving pictures. Consequently, the data files created containing these ECHO images are typically large data files, thus requiring necessary processing power.

All data collected is then passed to a processing system, for packaging into one comprehensive report. The processing system will include several tools necessary for the creation of the report, including a picture archiving and communication system (PACS). As will be outlined below, this report may be formatted in different manners, however, will universally contain all the different data types outlined above. Specifically, the report will be an electronic file containing clinical data, ECG data, and ECHO data all presented in a user friendly manner for easy access and review by cardiologists. This report will be saved within the PACS system, and available for access by other systems.

When access is desired, a cardiologist, or other specialist will access the processing system over a network such as the internet, for further review and interpretation. Using appropriate password schemes, the cardiologist will also be allowed to edit and revise the report, thus addressing any errors or inconsistencies while also providing the ability to add additional insight, based upon the review of the information. In this manner, a comprehensive cardiology report is created and maintained for use by virtually any healthcare provider, who would benefit from a review of the information. While it is anticipated that cardiologists will be the most significant user of these reports, other healthcare providers may also benefit.

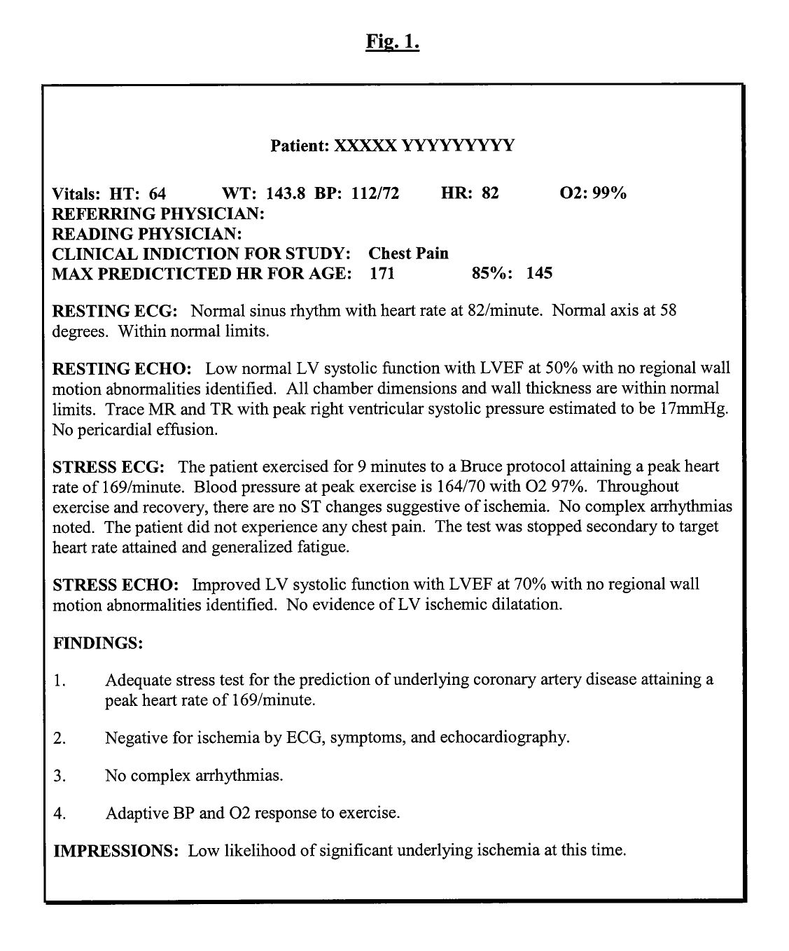

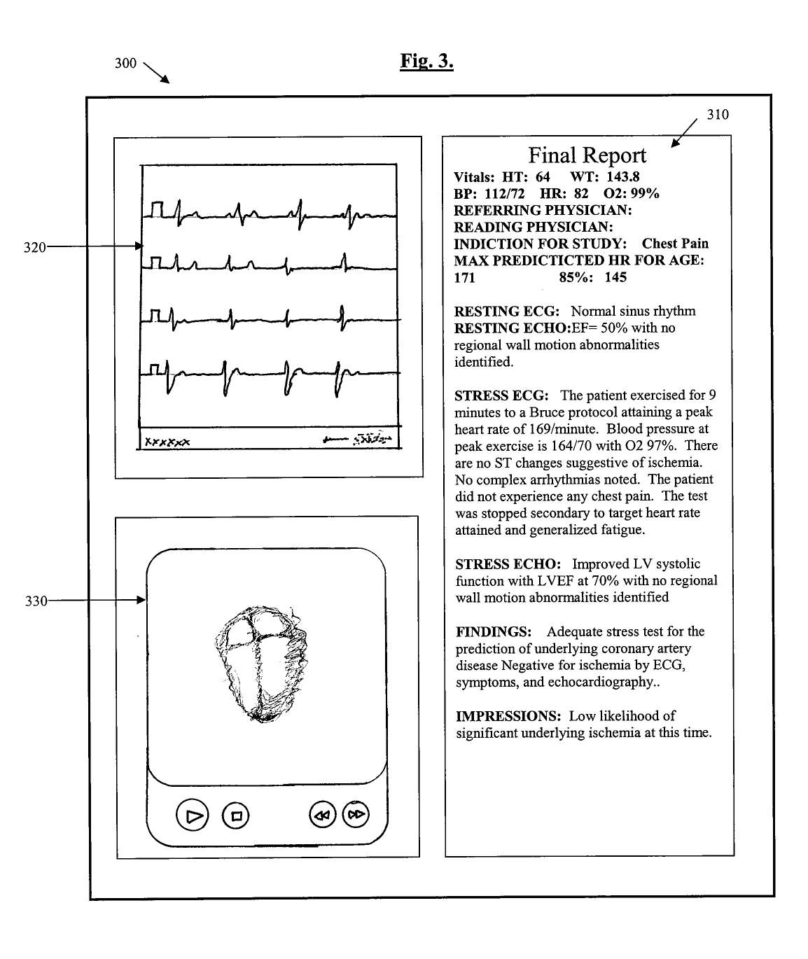

Referring now to FIG. 3 there is one embodiment of the final report presentation, as displayed on a users computer screen. As generally discussed above, this report includes three specific data types, including clinical data, ECG data and ECHO data, all presented in a user friendly format. Referring specifically to FIG. 3, a display 300 illustrates a number of data fields. These data fields include clinical data field 310, ECG data field 320 and ECHO data field 330. In this presentation, the clinical data field 310 includes previously entered data related to observations and clinical data, as traditionally carried out in a clinical data report. This information is essentially identical to the information contained in the report illustrated in FIG. 1. That said, this clinical data is presented here in a limited field and coupled with additional information.

ECG data field 320 provides an area for the presentation of graphical ECG data. ECG data field 320 can preferably be manipulated to allow movement and zooming of the graphical images illustrated. This could be carried out using many different graphic and document presentation programs. For example, the image may be incorporated into the report as a .pdf file which is viewable using an Adobe Acrobat viewer, which is well known.

In addition to the information outlined above, the display 300 includes an ECHO field 330, capable of displaying ECHO data in an appropriate format. As contemplated, this ECHO data is captured and best viewed in a video format, thus an appropriate video player will be implemented at this point. For example, video systems of the PACS system may be incorporated. Alternatively, Windows Media Player, Quick Time Video players, or other appropriate video players can be incorporated at this location of the display. In a preferred embodiment, the use of an embedded video player will allow the cardiologist to stop, freeze, fast-forward, rewind, etc. as necessary.

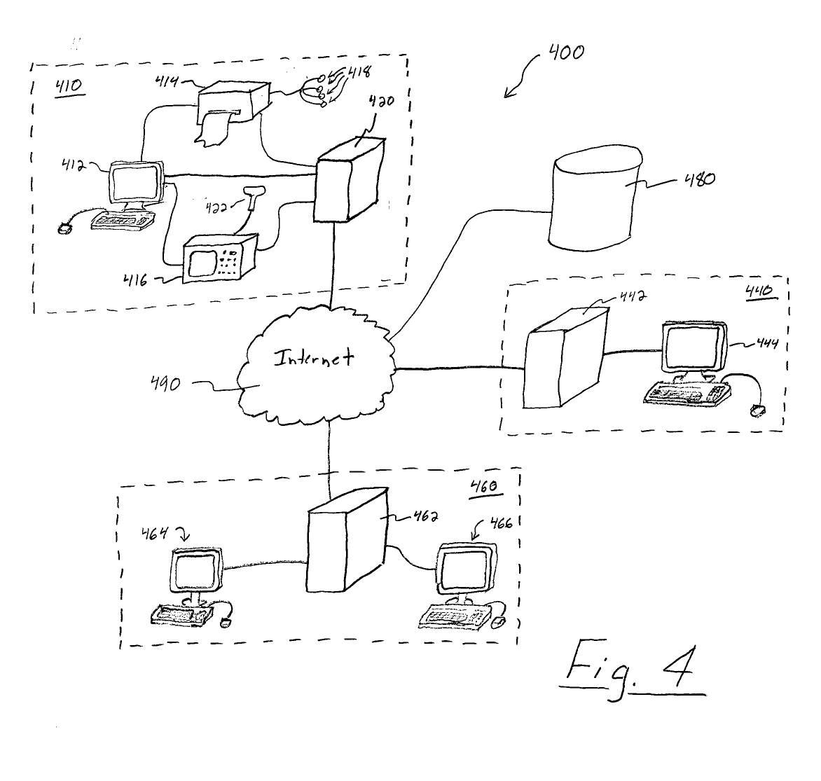

Referring now to FIG. 4, there is a schematic illustration of one potential system of the present invention. More specifically, overall data presentation system 400 is illustrated. In this embodiment, system 400 includes computer resources at three different locations, in addition to a designated offsite data storage device 480. More specifically, a first facility 410 includes a user station 412 coupled to an ECG system 414, an ultrasound system, 416 and a data processing system 420. In this case, the computer workstation or user station 412 will include typical PC components, such as a processor coupled to a monitor, keyboard, pointing device (mouse) etc. As can be anticipated, the user station 412 is utilized for data input, primarily generating the clinical data discussed above.

ECG system 414, will include appropriate leads and connections 418 which can be attached to a patient during ECG testing. A graphical output may be printed in a paper format, and/or may be generated in computer readable format. ECG machine 414, in this embodiment is coupled to both the user station 412, and the processing system 420.

Ultrasound device 416 has an appropriate ultrasound probe 422 for the collection of ECHO data during testing. Ultrasound device 416 is also coupled to both user station 412 and processing device 420. Upon receipt of all appropriate data, processing device 420 is used to collect the data in appropriate formats, and transform it to the desired report. This report will include all the above mentioned data types, in the desired format. It is understood that processing device 420 includes the same capabilities as the above mentioned PACS. At this point, processing device 420 is capable of transmitting appropriate data to other devices when necessary.

Also illustrated in FIG. 4 are remote access sites 440 and 460. Remote access site 440 includes a remote processing system 442 coupled with a user terminal 444. Similarly, a second remote access site 460 includes a second remote processing device 462, coupled to a first remote user terminal 464 and a second remote user access terminal 466. It is contemplated that first remote access site 440 and second remote access site 460 are virtually identical, with the exception of second remote accessing site 460 having two user terminals as opposed to one.

FIG. 4 shows each of these various sites connected via the internet 490 to provide appropriate communication of information. In one embodiment the present invention, the data collection site 410 may be located in a primary care physicians office for the collection and generation of all appropriate data. A cardiologist may be situated at either first remote access site 440 or second remote access site 460, and through the described connections, may be able to appropriately access the generated reports when necessary. Alternatively, data collection site 410 may be a dedicated testing site, while the PCP is located at first remote access site 440 and a cardiologist is located at second remote access site 460.

As illustrated in the drawings and discussion above, the system and methods of the present invention provide a valuable tool allowing users to access comprehensive patient data. In this context, patient data is formatted in a manner to provide a useful and valuable insights to cardiologists and primary care physicians.

The present invention is described above in reference to the drawings and certain embodiments. Foregoing detailed description and examples had been given for clarity of understanding the invention. Those skilled in the art will recognize that many changes can be made to the described embodiments without departing from the scope and spirit of the invention. Thus, the scope of the present invention should not be limited to the exact details and structures described herein, but rather the appended claims and equivalence thereof.

What is claimed is:

1. A system in communication with cardiovascular data sources for the compilation and comprehensive presentation of cardiovascular data, comprising:
a storage system for receiving and storing patient data including clinical data, electrocardiograph data, and echocardiogram data;
a processor coupled to the storage system for processing the patient data to generate a patient report wherein the patient report includes a display presentation capable of simultaneously presenting to a user the clinical data, electrocardiograph data and the echocardiogram data; and
a display coupled to the processor for presenting the display presentation to a user.

2. The system of claim 1 wherein the clinical data is text data, the electrocardiograph data is graphic data, and the echocardiogram data is video data.

3. The system of claim 2 wherein the patient report is a multimedia report capable of displaying the clinical data in a predetermined text format, displaying the electrocardiograph data in a graphical format, and playing the echocardiogram data as video.

4. The system of claim 1 wherein the display and processor are coupled to one another across a network.

5. The system of claim 4 wherein the network includes the internet.

6. The system of claim 1 wherein the display presentation is stored in the storage system as a display file, wherein the processor is further capable of transmitting the display file to a remote system capable of displaying the display presentation.

7. The system of claim 6 wherein the remote system is coupled to the processor via the internet.

8. The system of claim 7 wherein the patient report is a multimedia report capable of displaying the clinical data in a predetermined text format, displaying the electrocardiograph data in a graphical format, and playing the echocardiogram data as video.

9. The system of claim 1 wherein the patient report includes addition fields for use by the user, wherein the user is capable of adding to the patient report within the addition fields.

10. The system of claim 9 wherein the addition fields are capable of receiving text information from a user who is a cardiologist and wherein the text information includes analysis and diagnosis data.

11. The system of claim 1 wherein the clinical data, electrocardiograph data, and echocardiogram data are received from differing sources.

12. A method for the collection and comprehensive presentation of cardiovascular data for a patient, comprising:
collecting and storing clinical data for the patient;
collecting and storing stress electrocardiogram (ECG) data produced during a stress test of the patient;
collecting and storing stress echocardiogram (ECHO) data collected during a stress test of the patient;
integrating the clinical data, ECG data and the ECHO data with a computer processor to produce integrated patient report data which is capable of being transferred over the internet;
displaying to a physician the integrated patient data, thus allowing the physician simultaneous access to the clinical data, ECG data and ECHO data for the physician's interpretation and report.

13. The method of claim 12 wherein the integrated patient data is presented as a multimedia report capable of displaying the clinical data in a predetermined text format, displaying the electrocardiograph data in a graphical format, and playing the echocardiogram data in a video format.
